(19) United States
(12) Patent Application Publication
(10) Pub. No.: US 2005/0197542 A1
(43) Pub. Date: Sep. 8, 2005

(54) Method and a system for determining a physical condition, in particular the apparent age, of at least one region of the body or the face of an individual

(75) Inventors: Roland Bazin, Bievres (FR); Sophie Veyrat, Versailles (FR); Catherine Ressayre, Saint-Louis (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/044,457

(22) Filed: Jan. 28, 2005

Related U.S. Application Data
(60) Provisional application No. 60/556,369, filed on Mar. 26, 2004.

(30) Foreign Application Priority Data
Jan. 29, 2004 (FR) 04 50168

(51) Int. Cl. G06F 19/00; G01N 33/48; G01N 33/50

(52) U.S. Cl. 600/300; 702/19

(57) ABSTRACT

The present invention provides a method of evaluating a physical condition or a magnitude representative thereof. The method comprising: obtaining data for quantifying at least a first body state and a second body state that are different; and evaluating the physical condition or a magnitude representative of the physical condition by applying weighting coefficients or a segmentation tree to the data as obtained in this way. The coefficients or the tree are previously determined by statistical analysis on a group of reference individuals.

This non provisional application claims the benefit of French Application No. 04 50168 filed on Jan. 29, 2004 and U.S. Provisional Application No. 60/556,369 filed on Mar. 26, 2004.

The present invention relates to methods and systems suitable for use in determining a physical condition, for example the apparent age, of at least one region of the body or the face of an individual.

### BACKGROUND OF THE INVENTION

It is known that there exists a degree of correlation between the more or less wrinkled appearance of the face and real age. A chart with real age plotted along the abscissa and wrinkle evaluation scores plotted up the ordinate was presented at the 12th EADV Congress at Barcelona on Oct. 15 to 18, 2003 under the title “Clinical evaluation of the anti-wrinkle efficacy of retin-Ox correxion™” and is reproduced in FIG. 1.

U.S. Pat. No. 6,501,982, the content of which is incorporated in the present application by reference, describes a non-invasive system for estimating the relative age of an individual, the system comprising a spectrometer for measuring the radiation reflected by the skin in the near infrared. Such a system is relatively complex and expensive.

There exists a need to have available a method and a system enabling the apparent age of an individual to be determined without necessarily making use of equipment that is complex and expensive.

There also exists a need to be able to quantify in a manner that is as objective as possible the efficacy of a substance, in particular an anti-wrinkle substance, in terms of apparent rejuvenation.

Although the present invention may obviate one or more of the above-mentioned needs, it should be understood that some aspects of the invention may not necessarily obviate one or more of those needs.

In the following description, certain aspects and embodiments will become evident. It should be understood that the invention, in its broadest sense, could be practiced without having one or more features of these aspects and embodiments. It should be understood that these aspects and embodiments are merely exemplary.

### SUMMARY OF THE INVENTION

In one of its aspects, the invention may provide a method comprising:
obtaining data enabling at least first body state and a second body state of an individual to be quantified; and
evaluating a physical condition, for example apparent age, or a magnitude representative of said physical condition, by applying weighting coefficients or a segmentation tree to the data as obtained in this way, the coefficients or the tree being previously determined by statistical analysis on a group of reference individuals.

The term “body state” is used to designate a state of the body and/or the face, e.g. a state that is observable to the naked eye.

The term “data” should not be understood in a descriptive manner and covers alphanumeric values, e.g. decimal or binary values, whether or not they can be understood directly by an individual. The data may be conveyed by electrical or optical signals, whenever a body state is evaluated by using an instrument that delivers useful information in digital or analog form.

The apparent age that is evaluated may be a single value or an age range or it may be expressed relative to the real age of the individual being evaluated by words such as “appearance younger than real age” or “appearance older than real age”. Similarly, the magnitude representative of apparent age may be a single value or a range or it may express a difference relative to a reference value for a given real age.

The invention may make it possible to determine apparent age in relatively objective manner, with quantification of the first and second body states possibly being performed, for example, by means of an atlas and/or instruments, and thus in a manner that is not entirely subjective. Where appropriate, this quantification may be performed by self-evaluation by the individual concerned.

The invention also may make it possible to avoid having recourse to use of complex instruments, where appropriate.

In an exemplary implementation of the invention, apparent age may be evaluated by calculating an aging index.

Each of the first and second body states may be evaluated in at least two different regions, e.g. of the face.

At least one of the first and second body states may be an apparent sign of aging, observable to the naked eye without using an optical instrument.

Where appropriate, both of the first and second body states may be apparent signs of aging.

The first body state may be a characteristic relating to the relief of the skin, for example the formation of wrinkles, in particular on the skin and/or the hands.

The second body state may be a characteristic relating to the mechanical properties of the skin, e.g. its firmness.

The aging index may be determined from a combination, and in particular a linear combination, of a firmness index and a wrinkle index, for example.

The wrinkle index may be calculated from at least one combination, and in particular a linear combination, of wrinkle scores from different parts of the face. At least one of the scores may relate to wrinkles on the forehead, crow's foot wrinkles, wrinkles between the eyebrows, fine lines between the eyebrows, wrinkles under the eye, wrinkles at the corner of the mouth, the nasogenian folds, wrinkles of the upper lip, and wrinkles of the neck. The wrinkle index may comprise a linear combination of at least two such wrinkle scores.

The firmness index may be calculated from at least one combination, in particular a linear combination, of firmness scores, e.g. relating to different portions of the face and/or the body. The firmness index may be calculated from at least one score relating to sagging of the chin, to slackening of the neck, or to bags under the eyes. The firmness index may comprise a linear combination of at least two such firmness scores.

The wrinkle index and the firmness index may be calculated by combinations that are linear or non-linear.

Apparent age can be determined on the basis of the aging index by entering the value of the aging index into a graph or a table giving apparent age as a function of aging index, for example, where such a graph or table can be obtained by a panel evaluating a group of reference individuals that have also been subjected to the above-mentioned statistical analysis, for example.

The statistical analysis may comprise multivariate analysis, in particular by a partial least squares (PLS) method on the group of reference individuals. This produces a calibration data set, e.g. a set of wrinkle and firmness scores. The PLS method makes it possible to establish a regression model between at least one estimated variable said to be dependent or latent, and variables that are said to be independent or manifest and that explain the variations in the latent variable.

A history of the PLS method, including numerous bibliographic references is given in the article “Notes on the history and nature of partial least squares (PLS) modelling” by Paul Geladi, published in Journal of Chemometric, Vol. 2, 231-246 (1988), which article is hereby incorporated by reference.

When the apparent age or a magnitude representative of apparent age is calculated using a segmentation tree, the tree may be determined by an algorithm for discrimination by means of a binary decision tree. By way of example, the segmentation tree may comprise at least two tests relating to data obtained by evaluating body states, for example at least four tests relating to wrinkle scores and two tests relating to firmness scores.

A first applied test may relate, for example, to a score for crow's foot wrinkle depth. A second applied test may relate, for example, to a score either for wrinkles between the eyebrows or on the upper lip, depending on the result of the preceding test. When the second applied test relates to the score for the upper-lip wrinkles, the third applied test may relate to a score either for wrinkles under the eye, or for neck slackening, depending on the result of the second test. When the third test relates to the score for wrinkles under the eye, a fourth test may be applied relating to a score for sagging of the chin.

The invention may also provide a method of evaluating efficacy of a treatment, for example a cosmetic treatment, comprising:
using the above method to determine apparent age or a magnitude representative of apparent age, e.g. an aging index;
following a treatment;
determining apparent age or the magnitude representative of apparent age on a second occasion; and
evaluating the efficacy of the treatment, at least by comparing the apparent age or the magnitude representative of apparent age before and after treatment.

Such a method of evaluation may be useful for quantitatively evaluating the efficacy of a substance, in particular an anti-wrinkle substance, in terms of apparent age, e.g. in terms of change in aging index and/or apparent years of age.

The invention may also provide a method of promoting the sale of a treatment, in which reliance is placed on apparent rejuvenation revealed by a change in apparent age or a magnitude representative of apparent age, e.g. an aging index determined by the above method. Such promotion may be performed using any sales channel, e.g. by a sales person directly at a point-of-sale, by radio, by television, or by telephone, in particular in the form of commercials, short messages (SMS), or multimedia messages. It may also be performed via the written press or any other document, in particular for advertising purposes. It may also be performed over the Internet, or by any other suitable computer network or mobile telephone network. The promotion may also take place by mentioning the rejuvenation properties conferred by the substance directly on its packaging or on any instructions associated therewith.

The invention may also provide a system of evaluating a physical condition, for example the apparent age, of an individual, or a magnitude representative of said physical condition, the system comprising:
at least one atlas or instrument enabling a first body state to be quantified, for example by means of a score;
where appropriate, at least one atlas or instrument enabling a second body state to be quantified, for example by at least one score; and
calculation means for evaluating the physical condition or a magnitude representative thereof, by applying weighting coefficients or a segmentation tree to the data that serves to quantify the first and second body states, said coefficients or said tree being previously determined by statistical analysis of a group of reference individuals.

The weighting coefficients may be calculated by a PLS method on the basis of a calibration data set coming from a group of reference individuals, the individual whose physical condition is to be evaluated not necessarily belonging to said group.

The calculation means may comprise, for example, a microcomputer, a remote server, or any other computer equipment, charts, graphs, or any mechanical calculator, e.g. including a cursor or a rotary disk.

The invention also provides a kit for evaluating apparent age or a magnitude representative of apparent age, the kit comprising:
evaluation means enabling an individual to evaluate at least two different body states; and
calculation means enabling scores that result from evaluating body state to have weighting coefficients or a segmentation tree applied thereto, where the coefficients or the tree are previously determined by statistical analysis.

By way of example, the kit may be in the form of a computer medium containing a program serving firstly to display images and/or questionnaires for use in evaluating body states, and secondly to calculate apparent age or a magnitude representative of apparent age as a function of the evaluation of the body states. The computer medium may be constituted, for example, by a memory chip, an optical or magnetic disk, or any other medium.

The kit may also be in the form of at least one printed atlas and a computer medium. The printed atlas may then constitute all or part of the evaluation means and the computer medium may include at least one program enabling apparent age or a magnitude representative thereof to be calculated as a function of the evaluation performed using the atlas(es).

The kit may also include at least one atlas or other evaluation means and information enabling a connection to be made to a remote server, e.g. an Internet site address, the server being arranged, for example, to calculate the apparent age as a function of data obtained by using the evaluation means.

The invention may also provide a computer network site configured:
to enable an individual to evaluate at least two different body states and/or to enter data relating to the evaluation of said body states; and
to evaluate a physical condition, in particular apparent age or a magnitude representative of said physical condition, by implementing a method as defined above.

### BRIEF DESCRIPTION OF THE DRAWINGS

Aside from the structural and procedural arrangements set forth above, the invention could include a number of other arrangements, such as those explained hereinafter. It is to be understood that both the foregoing description and the following description are exemplary.

The accompanying drawings, which are incorporated in and constitute a part of this specification, illustrate a number of non-limiting embodiments of the invention and, together with the description, serve to explain the principles of the invention.

FIG. 1, described above, shows the state of the art;

FIG. 2 is a diagram showing the 95% confidence interval of the wrinkle index as a function of age range;

FIG. 3 is a diagram similar to FIG. 1 relating to the firmness index;

FIG. 4 shows apparent age and real age as a function of the aging index;

FIG. 5 is a diagram of an example of a comparison atlas;

FIG. 6 is a diagram of an example of an evaluation system including a computer;

FIGS. 7 and 8 show the use of morphing software respectively for evaluating chin sag and crow's foot wrinkles; and

FIG. 9 shows a segmentation tree for calculating the aging index.

### MORE DETAILED DESCRIPTION

In one examplary implementation of the invention, in order to determine the apparent age of an individual, an aging index iᵥ is calculated from a wrinkle index iᵥ and a firmness index i_{f}.

In order to obtain calibration data for calculating the above indices by means of a regression algorithm, wrinkles and firmness are evaluated on a group of reference individuals comprising a large enough number of individuals to be statistically representative of a given population. By way of example, 150 women are selected, with 30 women in each 10-year age range from 25 to 65 or more, and without any disease that might influence observations.

In order to calculate the wrinkle index iᵣ, it is possible, for example, to evaluate wrinkles using an evaluation atlas, by giving respective scores r₁ to r₁₀ for various categories of wrinkles, with a higher score being indicative of wrinkles that are more severe:
score r₁ from 0 to 5 for wrinkles of the forehead;
score r₂ from 0 to 4 for number of crow's foot wrinkles;
score r₃ from 0 to 6 for depth of crow's foot wrinkles;
score r₄ from 0 to 5 for wrinkles between the eyebrows;
score r₅ from 0 to 5 for fine lines between the eyebrows;
score r₆ from 0 to 5 for wrinkles under the eye;
score r₇ from 0 to 6 for wrinkles at the corner of the mouth;
score r₈ from 0 to 5 for the nasogenian folds;
score r₉ from 0 to 6 for wrinkles of the upper lip;
score r₁₀ from 0 to 5 for folds of the neck.

Each atlas has a set of comparison images corresponding to different grades for the wrinkles in question, and each image is associated with a score. Images are selected in relatively objective manner by comparing the person under evaluation with the images in the atlas.

Naturally, the invention is not limited to the above ten categories of wrinkles. Certain categories of wrinkles need not be quantified. In a variant, other categories of wrinkles may be added to the above list, e.g. marks on the back of the hand. The limit values for each score may be modified, where appropriate.

In order to calculate the firmness index i_{f}, it is possible to evaluate the following scores:
score f₁ from 0 to 4 for sagging of the chin;
score f₂ from 0 to 5 for slackening of the neck;
score f₃ from 0 to 5 for bags under the eyes.

The scores f₁ to f₃ may be evaluated by means of an atlas, in a manner similar to evaluating wrinkles.

Furthermore, for the individuals in the reference group, apparent age Aₐₚₚₐᵣₑₙₜ is evaluated using a panel made up of a plurality of men and women, e.g. 16 men and 16 women, including 16 people aged 22 to 30 and 16 people aged 35 to 64.

The wrinkle index iᵣ and the firmness index i_{f} constitute latent variables that can be expressed in the form of a linear combination of the wrinkle and firmness scores, which constitutes manifest variables, each of said scores having a weighting coefficient associated therewith, with the value of the weighting coefficient being representative of the contribution of the associated score to estimating the index in question.

The weighting coefficients for the wrinkle scores rᵢ and the firmness scores fᵢ are determined by multivariate statistical analysis.

In an implementation of the invention, the weighting coefficients are determined by the PLS method in which the manifest variables are associated with latent variables by simple regression.

An example of the PLS method is described by M. Tenenhaus in “La régression PLS Théorie et Practique” [The theory and practice of PLS regression], published by Editions Technip, Paris (1998).

In the example under consideration, the structural linear equations of the causal model are given in the conventional notation as follows:
iᵥ=β₃₀+β₃₁iᵣ+β₃₂i_{f}+τ₃

The PLS method comprises estimating
Yⱼ=Σ\tilde{W}ⱼₕ(xⱼₕ\overline{x}ⱼₕ)
of the standardized latent variables (PSIⱼ-mⱼ), where \tilde{W} is the calibration matrix, PSI₁ is the wrinkle index iᵣ, and PSI₂ is the firmness index i_{f}, with mⱼ being the mean of the PSIⱼ, with mean=0 and standard deviation=1.

The weights wⱼₕ of the matrix \tilde{W} are estimated and the latent variables are calculated using SAS V8.2 or the LVPLS 1.8 (J. B. Lohmöller, 1987) software, for example, and the structural equations are estimated by multiple regression, with Student's t test to test the significance of the regression coefficient and estimate the coefficients for the causal model given above.

The following relationships are obtained:

$$\begin{matrix}{i}_{r} = 0.059 {r}_{1} + 0.0849 {r}_{2} + 0.1256 {r}_{3} + 0.0892 {r}_{4} + 0.0937 {r}_{5} + \\ 0.1001 {r}_{6} + 0.1242 {r}_{7} + 0.0807 {r}_{8} + 0.0981 {r}_{9} + 0.071 {r}_{10} \\ {i}_{r} = 5 - ( 0.2912 {f}_{1} + 0.3848 {f}_{2} + 0.2976 {f}_{3} ) \\ and \\ {i}_{v} = 3.94 - 0.787 {i}_{r} + 0.22 {i}_{r}\end{matrix}$$

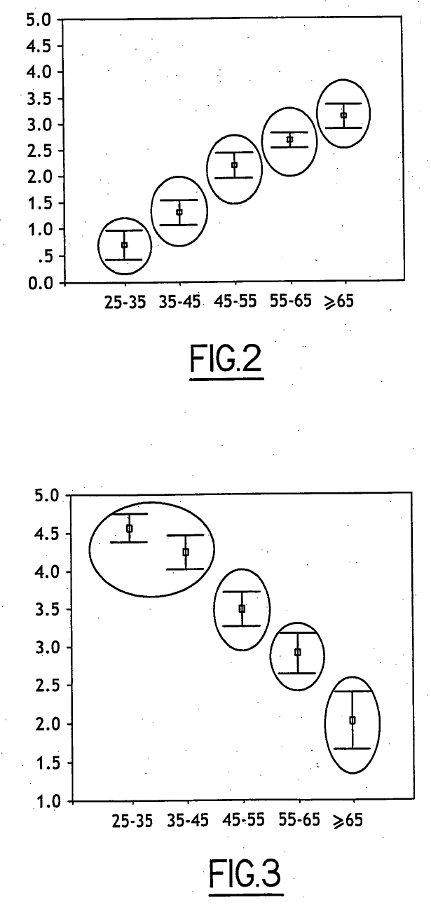

By calculating the wrinkle index iᵣ for the group of reference individuals and by proceeding, e.g. by means of SPSS V10.0 software, with ANOVA variance analysis over the monitoring ranges of a Tukey multiple comparison test, the graph of FIG. 2 is obtained, for example.

Age ranges are plotted along the abscissa and the wrinkle index iᵣ is plotted up the ordinate. The half-segments on either side of the bars correspond to the 95% confidence interval as a function of age range.

If the mean firmness index obtained in this way as a function of various real age ranges is compared by performing ANOVA variance analysis over the monitoring ranges of a Tukey multiple comparison test, then the results shown in FIG. 3 are obtained with age ranges plotted along the abscissa and firmness index up the ordinate, and with the half-segments representing the 95% confidence interval.

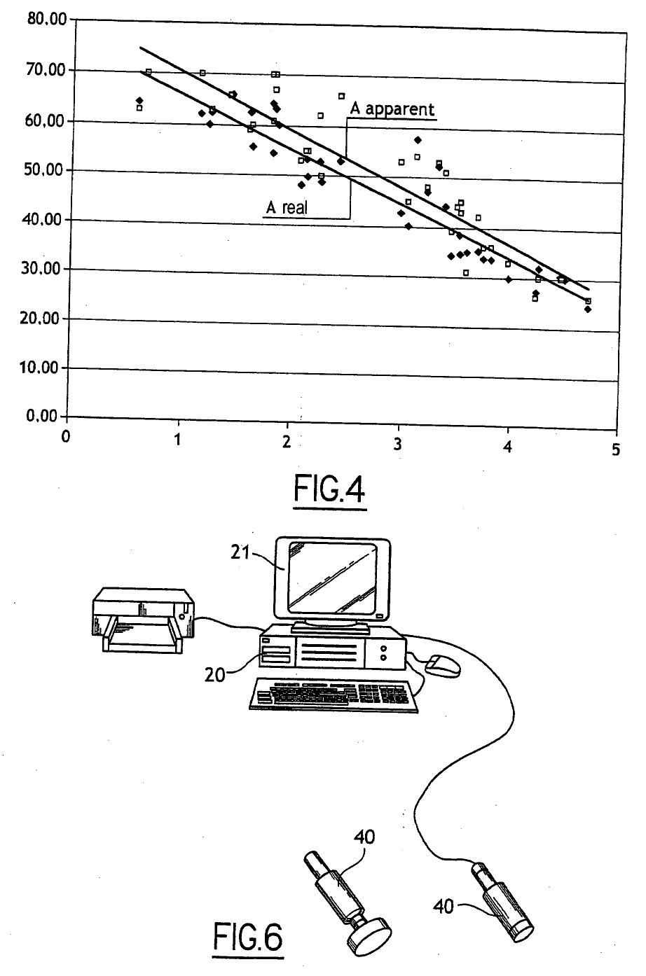

FIG. 4 shows a graph giving real age Aᵣₑₐₗ and apparent age Aₐₚₚₐᵣₑₙₜ as a function of the aging index iᵥ. By comparing the value of the aging index of an individual with a mean value observed for a corresponding age range in the group of reference individuals, it can be determined whether particular individuals appear older or younger than their real ages.

For a given individual, the aging index may also be evaluated other than by a linear combination of wrinkle scores and firmness scores.

For example, the aging index may be calculated using a segmentation tree determined using a CART method of discrimination by means of a binary decision tree, for example using SPAD V4.5 software, with the aging index iv as the continuous variable to be discriminated.

Those wrinkle and firmness variables that are the most discriminating when calculating the aging index can thus be identified.

In the example shown, this is the crow's foot wrinkle depth score r₃, the inter-eyebrow wrinkle score r₄, the chin sag score f₁, the upper-lip wrinkle score r₉, the score for wrinkles under the eye r₆, and the neck slacking score f₂.

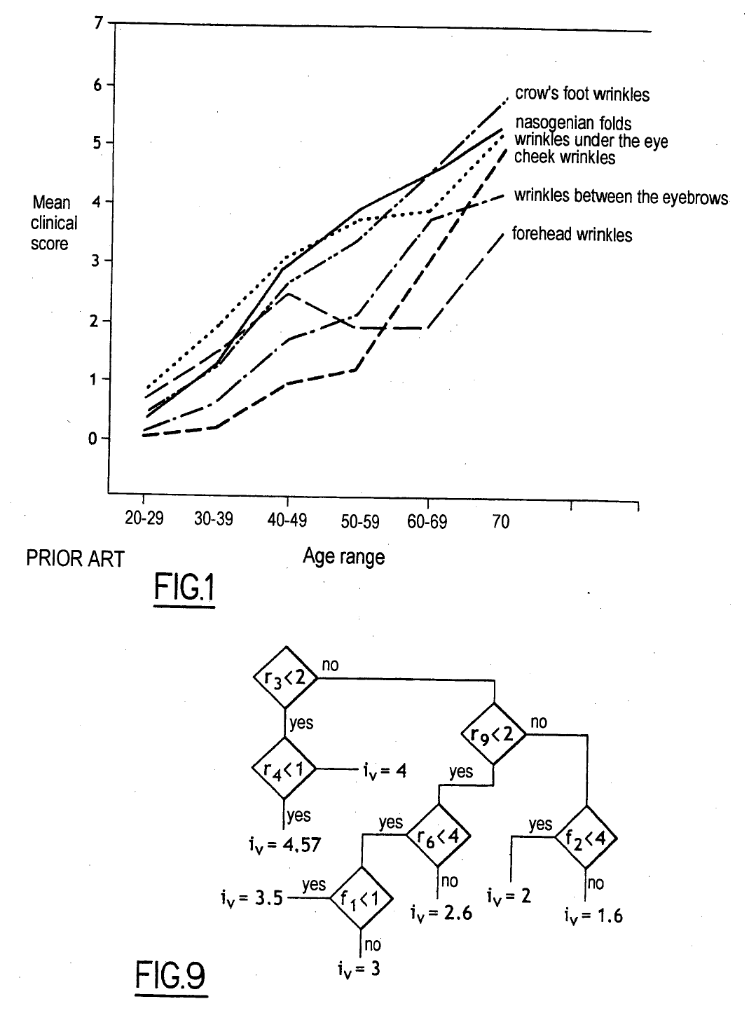

FIG. 1 gives the mean values of the aging index for each subgroup of individuals coming from the reference group and selected by the various tests.

It can be seen that the segmentation tree can serve to reduce the number of scores needed for determining the apparent age and/or the aging index, at least approximately.

Naturally, the invention is not limited to the examples described above.

For example, it is possible to evaluate a physical condition other than apparent age or to refine calculation of the aging index by introducing other manifest variables other than the various wrinkle or firmness scores, for example scores relating to quantity of white hairs, weight, quality of sleep, lack of appetite, blood pressure, cholesterol level, direction of skin lines, the size of pores in the skin, skin dryness, cellulite, skin elasticity, sweating, desquamation, uniformity of skin color, pigmentation marks, epidermal turnover, derm quality, sebum production, irritation, redness, enzymatic activity of the stratum corneum, this list not being limiting.

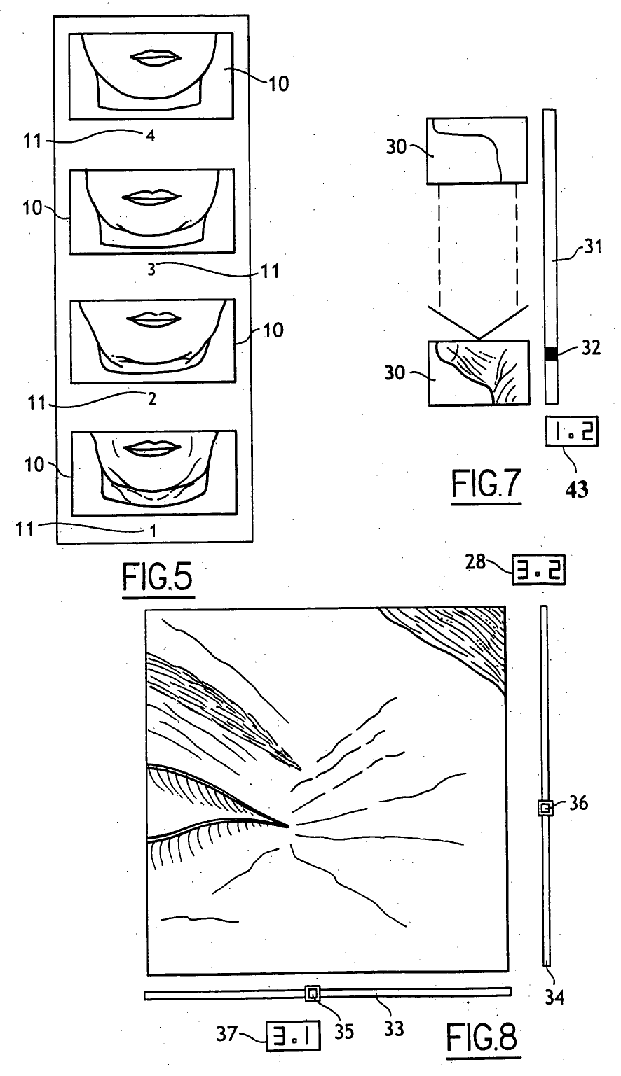

To evaluate a body state, it is possible to use an atlas, as mentioned above. By way of example, FIG. 7 shows an atlas comprising a plurality of comparison images 10 together with the corresponding scores 11.

It is also possible to use an evaluation system of the kind described in European patent application EP 1 216 658, the contents of which is incorporated herein by reference.

As shown in FIG. 8, such a system may comprise a computer 20 or any other computer equipment programmed to generate on a screen 21 a sequence of images 30 expressing different grades in a characteristic of body typology, for example sagging of the chin in the example of FIG. 7.

The system may display a scroll bar 43 with a cursor 32 for controlling scrolling of images from the sequence on the screen 21, with only one image of the sequence being displayed at a time.

The system may display an indicator 33 suitable for providing a score representative of the degree of the characteristic of body typology expressed by the image displayed on the screen 21.

Where appropriate, and as shown in FIG. 8, the system may generate two scroll bars 33 and 34 with two associated cursors 35 and 36, each cursor serving to control modification on the screen of a characteristic of the body typology expressed by the displayed image. Two indicators 37 and 38 are associated with each of the cursors 35 and 36 respectively. In the example shown, the cursor 35 may serve to modify the depths of wrinkles while the cursor 36 serves to modify the number of wrinkles in the crow's foot.

Where appropriate, various instruments 40 shown very diagrammatically in FIG. 6 can be used for quantifying body states, with at least one of the instruments being connected to the computer 20, where appropriate. These instruments may optionally deliver data directly to the computer 20 serving to estimate apparent age.

The instruments should be selected as a function of the body state that is to be evaluated.

By way of example, it is possible to use an instrument solely to determine the degree of a characteristic of body typology, as described in European patent application EP 1 277 437, the contents of which is incorporated herein by reference.

In order to quantify skin elasticity, it is also possible to use an evaluation tool as described in European patent application EP 1 277 436, the content of which is incorporated herein by reference. Such a tool may comprise two portions for application to the skin and interconnected by a connection enabling them to be moved relative to each other between a position in which the tool is placed on the skin, in which the two portions are close together, and a skin-stretching position in which the two portions are spaced apart, with the connection including at least one resilient member urging the two portions apart.

Where appropriate, at least one image of a non-dermatoglyphic zone of the skin may be acquired by means of an acquisition device comprising at least one non-optical sensor, serving to obtain information about the microrelief of said zone, in accordance with European patent application EP 1 177 766, the content of which is incorporated herein by reference. Such an acquisition device may comprise, in particular, a set of capacitive detection cells.

In order to evaluate the viscoelastic properties of the skin, it is also possible to use a device as described in French patent application No. 2 840 759, the content of which is incorporated herein by reference. Such a tool enables a temporary mark to be made on the skin and comprises a marker member for pressing against the skin, the marker member being configured in such a manner as to come into contact with the skin over a contact area that is not reduced to a point contact, said device further comprising means enabling the marker member to be applied against the skin with predetermined pressure. Over time, it is possible to observe the appearance of the mark and determine the time required for the mark to disappear for the naked eye or for the relief to smooth out past a predetermined threshold.

Where appropriate other instruments may be used, such as an elastometer, a corneometer, a motion picture camera, or a sebum meter.

In order to determine the apparent age of an individual, at least two body states, such as wrinkles and firmness for example, may be evaluated as described above by an adviser at a point-of-sale or in a beauty parlor. As a function of the results of the evaluation, treatment may be prescribed, such treatment comprising, for example, applying a substance, in particular a cosmetic, to the face.

In a variant, body states may be evaluated by people seeking to determine their own apparent age, e.g. by means of at least one atlas and/or any other instrument made available, in particular atlases for wrinkles and firmness. A person seeking to self-evaluate can thus give scores for wrinkles and firmness. The wrinkle index, the firmness index, and the aging and/or apparent age index can be calculated as a function of the values of the scores. The calculation may be performed, for example, by means of a computer medium supplied together with the atlas and/or the instrument, which medium is inserted into a personal computer by the person concerned. In a variant, the calculation may be performed by software that has been downloaded or by a program running on an Internet site, or any other server.

The atlases may be in printed form or they may be displayed as images on a screen, in particular as described in European patent application EP 1 216 658.

People seeking to undertake self-evaluation may connect to an Internet site, determine body states by means of a questionnaire including, for example, presenting an atlas, and subsequently find out their own apparent age and, where appropriate, have treatment prescribed seeking to rejuvenate their appearance.

The invention may also serve to evaluate the effectiveness of surgical and/or cosmetic treatment, where such treatment comprises, for example, applying a substance to the skin, in particular an anti-wrinkle agent, administering food supplements or medication, and/or following a specific diet and/or surgical acts, such as, for example, a facelift and/or injecting botulinum toxin (botox).

In order to evaluate the incidence of treatment on apparent age, the aging index can be calculated before and after the treatment. In the example of FIG. 4, a change in the aging index of 0.6 corresponds substantially to a reduction of ten years in apparent age.

Although the above description applies more particularly to the apparent age of the face, the invention is not limited to calculating the apparent age of the face and can be applied to the apparent age or some other physical condition of any other region of the body, in particular the bust or the legs, the manifest variables being selected accordingly. For example, they may be scores for bust wrinkles, breast firmness, cellulite, jodhpur thighs, skin type descriptions, for example greasy skin, dry skin, sensitive skin, photo-aged skin.

Throughout the description, including in the claims, the term “comprising a” should be understood as being synonymous with “comprising at least one” unless specified to the contrary.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of at least one of evaluating a physical condition and a magnitude representative of said physical condition, comprising:
obtaining data for quantifying at least
a first body state and
a second body state, the second body state being different from the first body state; and
evaluating at least one of physical condition and a magnitude representative of said physical condition by applying at least one of weighting coefficients and a segmentation tree to the data, the coefficients or the tree being previously determined by statistical analysis on a group of reference individuals.

2. A method according to claim 1, wherein the physical condition comprises apparent age.

3. A method according to claim 2, wherein the evaluation of apparent age comprises calculating an aging index.

4. A method according to claim 2, wherein each of the first and second body states is evaluated in at least two different regions.

5. A method according to claim 2, wherein the first body state relates to a skin relief characteristic.

6. A method according to claim 5, wherein the first body state relates to wrinkle formation.

7. A method according to claim 2, wherein the second body state relates to a mechanical characteristic of the skin.

8. A method according to claim 7, wherein the second body state relates to skin firmness.

9. A method according to claim 3, wherein the aging index is determined from a combination of a firmness index and a wrinkle index.

10. A method according to claim 9, wherein the aging index is determined from a linear combination of a firmness index and a wrinkle index.

11. A method according to claim 2, wherein a wrinkle index is calculated on the basis of at least one combination of wrinkle scores for different portions of the face.

12. A method according to claim 2, wherein a wrinkle index is calculated on the basis of at least one linear combination of wrinkle scores for different portions of the face.

13. A method according to claim 11, wherein a different portions of the face are selected among forehead wrinkles, crow's foot wrinkles, wrinkles between the eyebrows, fine lines between the eyebrows, wrinkles under the eye, wrinkles at the corner of the mouth, nasogenian folds, wrinkles on the upper lip, and neck wrinkles.

14. A method according to claim 9, wherein the firmness index is calculated from at least one combination of firmness scores relating to different portions of the face.

15. A method according to claim 14, wherein the combination is a linear combination.

16. A method according to claim 14, wherein the firmness index is calculated from scores for skin sagging, neck slackening, and bags under the eyes.

17. A method according to claim 3, wherein apparent age is determined from the aging index by entering the value of the aging index into at least one of a graph and a table giving apparent age as a function of the value of the aging index.

18. A method according to claim 1, wherein the statistical analysis comprises analysis by a PLS method on the group of reference individuals.

19. A method according to claim 1, wherein the segmentation tree is determined by an algorithm for discrimination by a binary decision tree.

20. A method according to claim 19, wherein the segmentation tree comprises a first test relating to a score for depth of crow's foot wrinkles, a second test relating to a score for at least one of wrinkles between the eyebrows, and for upper-lip wrinkles, depending on the result of the preceding test, and when the second applied test relates to the score for upper-lip wrinkles, a third test is applied that relates the at least one of a score for wrinkles under the eye and a score for neck slackening, depending on the result of the second test, and when the third test relates to the score for wrinkles under the eye, a fourth test is applied relating to a score for sagging of the chin.

21. A method of evaluating the effectiveness of a treatment comprising:
using the method as defined in claim 1 a first time to determine at least one of apparent age and a magnitude representative of apparent age;
applying a treatment;
determining at least one of apparent age and a magnitude representative of apparent age a second time; and
evaluating the effectiveness of the treatment by comparing the determinations before and after treatment.

22. A method according to claim 21, wherein the treatment comprises a cosmetic treatment.

23. A method according to claim 21, comprising determining an aging index.

24. A system for evaluating at least one of a physical condition of an individual and a magnitude representative of said physical condition, the system comprising:
at least one of an atlas and an instrument enabling at least a first body state to be quantified;
where appropriate, at least one additional atlas or instrument enabling at least one second body state to be quantified; and
calculation means enabling at least one of a physical condition and a magnitude representative of said physical condition to be evaluated by applying at least one of weighting coefficients and a segmentation tree to data resulting from having quantified the first and second body states, which coefficients or tree are determined by statistical analysis on a group of reference individuals.

25. A system according to claim 24, wherein the physical condition comprises apparent age.

26. A system according to claim 24, wherein the statistical analysis comprises a PLS method.

27. A system according to claim 24, wherein the segmentation tree is determined by an algorithm for discrimination by a binary decision tree.

28. A kit for evaluating at least one of apparent age and a magnitude representative of apparent age, the kit comprising:
evaluation means enabling an individual to evaluate at least two different body states; and
calculation means enabling at least one of apparent age and a magnitude representative of apparent age to be calculated by applying at least one of weighting coefficients and a segmentation tree to the data resulting from evaluating the body states, the coefficients or the tree being determined by statistical analysis on a group of reference individuals.

29. A kit according to claim 28, wherein the kit comprises a computer medium containing a program configured firstly to display at least one of images and questionnaires for evaluating body states, and secondly to calculate the apparent age or the magnitude representative of apparent age.

30. A kit according to claim 29, wherein the kit comprising at least one printed atlas and a computer medium, the printed atlas constituting at least part of the evaluation means and the computer medium comprising at least one program enabling at least one of apparent age and a magnitude representative thereof to be calculated.

31. A kit according to claim 29, further comprising information enabling a connection to be made to a remote server, the remote server being configured to calculate apparent age as a function of data obtained using the evaluation means.

32. A kit according to claim 31, wherein the remote server comprises an Internet site address.

33. A computer network site configured:
to enable an individual to at least one of perform self-evaluation of at least two different body states and input data relating to the evaluation of said body states; and
to evaluate at least one of a physical condition and a magnitude representative of said physical condition by implementing the method as defined in claim 1.

34. A computer network site according to claim 33, wherein the physical condition comprises apparent age.

35. A method of promoting the sale of a treatment, comprising:
placing reliance on apparent rejuvenation revealed by a change in at least one of apparent age and a magnitude representative of apparent age determined by the method as defined in claim 1.

36. A method according to claim 35, comprising mentioning rejuvenation properties conferred by a substance on at least one of packaging and instructions associated therewith.
